(19) United States
(12) Patent Application Publication
(10) Pub. No.: US 2026/0198804 A1
(43) Pub. Date: Jul. 16, 2026

(54) DEVICE AND METHOD FOR STIMULATING AT LEAST ONE OF A NERVE AND A MUSCLE OF A PATIENT HAVING A PATHOLOGICAL GAIT

(71) Applicant: KURAGE, Lyon (FR)

(72) Inventors: Baptiste MOREAU, Lyon (FR); Lana POPOVIC MANESKI, Belgrade (RS); Romaric NOLLOT, Grenoble (FR)

(73) Assignee: KURAGE, Lyon (FR)

(21) Appl. No.: 19/138,143

(22) PCT Filed: Dec. 15, 2023
(86) PCT No.: PCT/EP2023/086207

§ 371 Date: Jun. 12, 2025

(30) Foreign Application Priority Data
Dec. 15, 2022 (EP) 22306891.7

(51) Int. Cl. A61B 5/11; A61B 5/00; A61B 5/103; A61B 5/389; A61N 1/36

(52) U.S. Cl. CPC A61B 5/112; A61B 5/1038; A61B 5/1128; A61B 5/389; A61B 5/6807; A61B 5/7267; A61B 5/7278; A61N 1/36003; A61N 1/36034

(57) ABSTRACT

A device for training a personalized model of a gait cycle configured to generate a gait phase information representative of a state of advancement of a patient in the gait cycle. A device and method for computing a start timepoint and an end timepoint and an intensity of at least one sequence of electrical pulses to apply to at least one of a nerve and a muscle of a patient using at least one pair of electrodes, based on a gait phase information representative of a state of advancement of a patient in the gait cycle obtained using a personalized model of a gait cycle obtained from the device for training.

### FIELD

The present invention relates to the field of neuro-rehabilitation systems for stimulating the nerves and/or the muscles of a patient suffering from a pathological gait. More specifically, the invention related to a device for training a personalized model of a gait cycle configured to generate a gait phase information representative of a state of advancement of a patient in the gait cycle and to a device and method for computing a start timepoint and an end timepoint and an intensity of at least one sequence of electrical pulses to apply to at least one of a nerve and a muscle of a patient using at least one pair of electrodes.

### BACKGROUND

According to the World Health Organization, around 15 million peoples fall victim to stroke every year around the world, and this number is estimated to grow by 3.4 million people by 2030. Strokes are medical emergencies that impact the supply of blood in the brain. With appropriate and timely medical treatment, people's life is often saved, but potential brain damage will likely cause secondary effects.

The most outwardly notable effects of a stroke are the ones that impact physical movement. Many stroke survivors sustain motor impairments that affect one side of the body (the side opposite to where the stroke occurred). Hemiparesis describes weakness on one side of the body while hemiplegia describes paralysis on one side of the body.

As a consequence, the gait of some patients may be altered, which leads to an increased risk of injuries, for instance due to a fall or a repeated incorrect movement.

The gait of a patient corresponds to the style of walking. It is different from one patient to another and it may change during the patient's lifetime depending on his age, mood and general health state.

Overall, it is possible to define a gait cycle, which is the cyclic pattern of movement that occurs while walking. The gait cycle can be divided into stance phases and swing phases.

The stance phase corresponds to the period during which a first leg is touching the floor while the second leg is being swinged forward. During this phase, almost all the bodyweight of the patient rests on the first leg.

The swing phase corresponds to the period during which the first leg becomes free to move forward, while the second leg rests on the floor.

During the stance phase, it is possible to identify 5 stages. The initial contact or heel strike corresponds to the moment where the heel of the first leg makes initial contact with the ground. The loading response or foot flat is the moment where the foot rolls forward until the entire plantar surface is in contact with the ground. The mid stance starts when the second leg is propelled forward so that the entire body weight is being balanced over the first leg. The terminal stance or heel-off starts when the first leg heel start being lifted off the ground. This is when the body weight starts to shift onto the second leg. The pre-swing or toe-off is the final stage of the stance phase and includes pushing the toes into the ground, thus creating a forward propulsion.

The swing phase can be divided into 3 stages. The early swing or acceleration phase is the first stage during which the foot of the first leg is lifted from the ground. The ankle and knee flex so that the foot and toes can be moved from the ground. The hip flexes to bring the leg forward, moving it directly under the body. The mid-swing is when the first leg passes directly beneath the body and past the second leg bearing the weigh. At the same time, the trunk is moved forward so that the weight of the body is directly over the second leg. The late swing or declaration phase corresponds to the moment where the knee extends and the foot is approaching the floor. The first leg is now ready for the start of the next stance phase.

Treatment of pathological gait involves physical and occupational therapy to rewire or reestablish the neural connections within the brain and restore movement.

One other facet of treatment pertains to the exploration of compensatory techniques. As such, Functional Electrical Simulation (FES) for stroke patients offers a wide range of benefits and can help improving motor skills. Surface FES works by placing non-invasive electrodes on the skin. Once activated, these electrodes send electrical impulses to the nerves before the entry to the muscles, causing the muscles to contract.

Some hybrid solutions, for hemiplegic patients, consist of combining a motorized walking assistance system with a FES system. Combination of FES and robotic control is a challenging issue, due to the non-linear behavior of muscle under stimulation and the lack of developments in the field of hybrid control. In particular, coordination between the FES and the actual gait of the patient is difficult to obtain.

### SUMMARY

The invention thus relates to a device for training a personalized model of a gait cycle configured to generate a gait phase information representative of a state of advancement of a patient in the gait cycle, said device comprising:
at least one input configured to receive:
   for each subject of a plurality of subjects, subject motion data and subject foot plantar pressure data acquired during a plurality of gait cycles,
   for said patient, patient motion data and patient foot plantar pressure data acquired during a plurality of gait cycles,
      said subject foot plantar pressure data and patient foot plantar pressure data being representative of a strength of a contact of at least one region of a foot with a ground, said patient foot plantar pressure data and patient foot plantar pressure data being acquired with at least one pressure sensor placed under said foot,
at least one processor configured to:
   generate a generic training dataset based on said subject motion data and subject foot plantar pressure data from the plurality of subjects,
   generate a patient training dataset based on said patient motion data and patient foot plantar pressure data,
   train a generic model of a gait cycle using said generic training dataset so as to obtain a pre-trained generic model of a gait cycle, and
   retrain said pre-trained generic model of a gait cycle using said patient training dataset so as to obtain said personalized model of a gait cycle,
at least one output configured to provide said personalized model of a gait cycle.

Advantageously, the device of the present invention provides a model of a gait cycle that is personalized to the pathological gait of the patient. Indeed, every patient has its own specific pathological gait (e.g. depending on the type and stage of the disease and on many other factors such as the age, weight, or height) and no generic model of a gait cycle can be adapted precisely enough to the patient's gait using data coming from random subjects.

By training the generic model of a gait cycle using a dataset representative of the patient's specific gait cycle, the performances in precisely determining the state of advancement of a patient in the gait cycle are greatly improved. Therefore, thanks to the precision of the prediction of the personalized model of a gait cycle, it is possible to generate electrical stimulation that are very precise and adapted to correct the pathological gait of the patient. Indeed, if the generic model of a gait cycle is trained to recognize the specific gait cycle of the patient, it becomes more accurate in calibrating the timings and intensities of electrical pulses to simulate a healthy gait.

According to one embodiment, the training performed is a transfer learning method where a pre-trained model is re-used as the starting point for a model on a new task. To put it simply, the generic model of a gait cycle is trained to recognize generic gait phase information and it is repurposed to recognize the gait phase information specific to the patient. By applying transfer learning to a new task, one can achieve high performances with only a small amount of data. Compared to traditional machine learning models who require training from scratch, which is computationally expensive and requires a large amount of data to achieve high performance, transfer learning is computationally efficient and helps achieve better results using a smaller data set at a faster pace. The training of the generic model of a gait cycle is preferably performed using data coming from wearable motion sensors such as IMUs (Inertial Motion Units) positioned on the hip and leg segments of the patient and pressure sensors positioned under the feet of the patient. Pressure sensors and motion sensors are indeed smaller and more easily wearable that other sensors such as vision motion capture systems. They can easily be integrated in clothes or shoes. On the contrary, vision motion capture systems need to be positioned far from the patient to capture his movements, which is very unpractical to use on a regular basis. Besides, pressure sensors a give instantaneous contact information between the foot and the ground, so they can be relied upon to segment the gait between stance and swing phases.

According to other advantageous aspects of the invention, the device comprises one or more of the features described in the following embodiments, taken alone or in any possible combination.

According to one embodiment, generating said generic training dataset comprises, for each subject of the plurality of subjects, detecting on the subject foot plantar pressure data and subject motion data, a toe-off event for each gait cycle of the plurality of gait cycles, and associating said toe-off event with a pre-determined subject state of advancement in the gait cycle.

According to one embodiment, generating said patient training dataset comprises, detecting on the patient foot plantar pressure data and patient motion data, a toe-off event for each gait cycle of the plurality of gait cycles, and associating said toe-off event with a pre-determined patient state of advancement in the gait cycle.

According to the invention, the pre-determined subject state of advancement in the gait cycle and the pre-determined patient state of advancement in the gait cycle may be identical or different.

The gait phase information may for instance be expressed as a percentage comprised between 0 and 100. Thus, according to one embodiment, said pre-determined subject state of advancement in the gait cycle is a percentage of advancement in the gait cycle of 60%.

According to another embodiment, said pre-determined patient state of advancement in the gait cycle an is a percentage of advancement in the gait cycle of 60%.

Annotating the toe-off event consistently at a pre-determined state of advancement in the gait cycle (e.g. for example 60%) in the data used for training the generic model of a gait cycle can improve training in several ways.

It can help with temporal alignment across all instances in the dataset. This ensures that relevant features leading up to and following the toe-off event are consistently captured and included in the training data, thus facilitating the learning of patterns in the gait cycle across different individuals or conditions, leading to improved generalization to new, unseen data. Indeed, gait cycles can exhibit variability among subjects or under different conditions. By consistently annotating the toe-off event at a specific pre-determined state of advancement, the model becomes less sensitive to minor variations and can learn the essential features that characterize the toe-off event, irrespective of subjects'differences.

If the model is intended for use across different datasets or populations, consistent annotation improves the chances of successful transfer learning. The model trained on one dataset with consistent annotations is more likely to transfer well to another dataset with similar annotations.

The toe-off event is advantageously chosen for annotation because it marks a critical transition in the gait cycle. Annotating specifically the toe-off event allows great improvement in the predictions of the model compared to other events in the gait cycle.

According to other embodiments, generating said generic training dataset may also comprise, for each subject of the plurality of subjects, detecting on at least one of the subject foot plantar pressure data and subject motion data, a heel strike event for each gait cycle of the plurality of gait cycles, and associating said heel strike event with a transition point between two gait cycles.

The heel-strike event may be annotated at a second pre-determined state of advancement in the gait cycle. The second pre-determined state of advancement may be the starting point of the gait cycle. It may be expressed as a percentage of advancement in the gait cycle of 0%.

According to one embodiment, said generic model of a gait cycle is an oscillatory neural network (ONN).

ONN are designed to exhibit oscillatory behavior. The Applicant has found out that ONN are particularly suitable for modeling and capturing cyclical patterns like the gait cycle. The ONN architecture aligns well with the natural rhythmic nature of walking. Gait involves complex temporal dynamics, with distinct phases such as heel strike, midstance, toe-off, and swing. An ONN, with its oscillatory activations, can efficiently represent and capture these temporal dynamics. Moreover, the oscillatory nature of an ONN allows it to adapt to variable gait speeds. Since walking speeds can vary among subjects and compared to the patient, the ability of the ONN to naturally adjust its oscillations can contribute to better generalization across different walking conditions. Additionally, the oscillatory features learned by the ONN during training on one dataset may be transferable to other datasets with similar cyclic patterns. This makes the ONN well-suited for transfer learning scenarios where knowledge gained from the generic dataset that might have been acquired on subjects with a pathological gait or a healthy gait can be applied to the dataset acquired on the patient that has his own specific gait.

The oscillator type-model also facilitates real-time adjustments in the stimulation process, ensuring optimal muscle activation corresponding to the current phase of the gait cycle.

According to one embodiment, subject motion data and subject foot plantar pressure data are acquired on subjects having a pathological gait.

Using a generic training dataset acquired on subjects with a pathological gait surprisingly showed a significant improvement compared to using training sets from subjects with a normal gait. Indeed, one might think that a learning performed on subjects having a “normal gait” would lead to better results given the consistency of the dataset. However, on average, the MSE (Mean Square Error) decreased by 38%. This reduction in MSE indicates that training the generic model using subjects with a pathological gait achieves better accuracy in estimating gait phases compared to the using subjects with a normal gait.

According to another aspect, the invention relates to a device for computing a start timepoint and an end timepoint and an intensity of at least one sequence of electrical pulses to apply to at least one of a nerve and a muscle of a patient using at least one pair of electrodes, based on a gait phase information representative of a state of advancement of a patient in the gait cycle obtained using a personalized model of a gait cycle obtained from a device for training described above, wherein said device comprises:
at least one input configured to receive, for said patient, patient motion data and patient foot plantar pressure data acquired during a plurality of gait cycles, said patient foot plantar pressure data being representative of a strength of a contact of at least one region of a foot with the ground, said patient foot plantar pressure data being acquired with at least one pressure sensor placed under said foot,
at least one processor configured to:
   feed said patient motion data and patient foot plantar pressure data to said personalized model of a gait cycle and output a gait phase information representative of a state of advancement of a patient in the gait cycle,
   compute said start timepoint and an end timepoint and said intensity of said at least one sequence of electrical pulses to apply to at least one of said nerve or said muscle of said patient using said at least one pair of electrodes, and
at least one output configured to provide said start timepoint and end timepoint and intensity.

According to the invention, the nerves on which at least one sequence of electrical pulses may be applied are sensory nerves and/or motor nerves. Moreover, the at least one sequence of electrical pulses may also be applied on motor points of muscles, where nerve endings enter the muscle or on sensory receptors in the skin that are innervated.

Sensory nerves are a type of nerve that carries sensory information from the sensory organs, such as the skin, muscles, joints, and internal organs, to the central nervous system (CNS). These nerves play a crucial role in transmitting signals related to touch, temperature, pain, proprioception (awareness of body position), and other sensory modalities. Sensory nerves are responsible for gathering information about the external environment and the body's internal state, allowing the central nervous system to process and respond to these stimuli.

Motor nerves are a type of nerve that carries signals from the central nervous system (CNS) to the muscles. These nerves play a key role in controlling voluntary and involuntary movements. Motor nerves transmit signals that initiate muscle contractions. Motor nerves are essential for executing motor commands generated by the brain and spinal cord, enabling various bodily movements and functions.

The electrical charge threshold to stimulate nerves can vary depending on several factors, including the type of nerve (sensory or motor), the specific nerve fiber diameter, and individual variability. Additionally, different nerves may have different excitability thresholds. As a general rule, motor nerves often have higher excitation thresholds than sensory nerves. Hence, to stimulate a patient at a sensory level, the intensity of the at least one sequence of electrical pulses may be comprised between zero and a first charge threshold. Electrical charge is determined by the current intensity and duration of the stimulation pulse, as well as by the frequency of pulses. To stimulate a patient at a motor level, the intensity of the at least one sequence of electrical pulses may be comprised between the first charge threshold and a second charge threshold.

Advantageously, the device is capable of computing in real time the sequence of electrical pulses to apply to the nerves and/or muscles of the patient so that the patient can have his gait adjusted almost at very moment he initiates a voluntary contraction.

In practice, feeding said patient motion data and patient foot plantar pressure data to said personalized model of a gait cycle comprises using a rolling window.

This feature allows to consider a plurality of gait cycles of the patient and to get a better idea of the actual patient gait. Indeed, the data may be averaged on the plurality of gait cycles to smoothen out sensors inaccuracy and events that are not systematically present on the patient's gait.

According to one embodiment, said at least one input is further configured to receive patient environmental data representative of at least one parameter of an environment of said patient, said at least one processor being further configured to feed said patient environmental data to said personalized model of a gait cycle in order to adapt the computing of the start timepoint and end timepoint and of the intensity of the at least one sequence of electrical pulses as a function of the at least one parameter of the environment of said patient.

According to one embodiment, said at least one parameter of an environment of said patient is at least one of a presence, a distance and a size of at least one obstacle present in said environment.

According to the invention, obstacles refer to a physical entity on the way that hinders or obstructs progress in the gait. It may also be a change in terrain (e.g. stairs, inclination, different ground material such as sand).

To that end, the patient may be equipped with sensors such as ultrasound distance sensors and/or cameras to detect obstacles on the way, shape and distance of the obstacle to adapt the personalized model of a gait cycle for delivering sequences of pulses to the muscles to avoid falls.

According to one embodiment, said at least one input is further configured to receive patient EMG data, said patient EMG data being representative of at least one of a muscle activity, a muscle spasticity or a muscle spasm, said at least one processor being further configured to feed said patient EMG data to said personalized model of a gait cycle in order to adapt the computing of the start timepoint and end timepoint and of the intensity of the at least one sequence of electrical pulses as a function of the patient EMG data.

According to the invention, the muscle activity refers to the contraction and relaxation of muscles, that result in voluntary movements. Monitoring and measuring muscle activity (e.g. intensity, frequency and duration of muscles contractions) can be done using electromyography (EMG), a technique that records the electrical activity produced by muscles during contraction and relaxation.

According to the invention, muscle spasticity refers to a continuous state of increased muscle tone or stiffness compared to normal. Muscles affected by spasticity exhibit heightened resistance to passive stretching. They may feel rigid or stiff. The higher the spasticity, the higher the stiffness. Muscle spasticity can be deduced by comparing an electromyography (EMG) signal of a patient suffering from muscle spasticity with signals coming from healthy subjects.

According to the invention, muscle spasms are sudden involuntary tightening or contraction of a muscle. These contractions can be brief or prolonged and may involve a single muscle or a group of muscles.

In other words, the patient EMG data may be inversely correlated with the intensity of the at least one sequence of electrical pulses. The stronger a voluntary contraction of at least one muscle of said patient is detected via the patient EMG data, the lower the intensity of the at least one sequence of electrical pulses should be. If the voluntary contraction of at least one muscle of said patient is above a pre-determined threshold, the intensity of the at least one sequence of electrical pulses may even stimulate said patient's nerve at a sensory level.

To that end, said at least one input may be further configured to receive patient EMG data, said patient EMG data being representative of at least one of a muscle activity, a muscle spasticity or a muscle spasm, said at least one processor being further configured to feed said patient EMG data to said personalized model of a gait cycle in order to determine if said at least one sequence of electrical pulses should be delivered to at least one of said nerve and said muscle at a motor level or at a sensory level.

According to one embodiment, said at least one sequence of electrical pulses is delivered at a motor level when said patient EMG data exceeds a pre-determined value.

Said pre-determined value may be representative of at least one of an intention and a strength of a voluntary action performed by said patient. Moreover, the pre-determined value may be representative of an activity that is similar to the activity of the antagonist muscle.

Such pre-determined value may be decided by the therapist depending on the patient and on the severity of his pathological gait.

The invention further relates to a system for stimulating a muscle of a patient, comprising:
a multi-channel electrostimulator comprising at least one pair of electrodes,
at least one inertial measuring unit configured to generate said patient motion data and at least one pressure detection unit configured to generate said patient foot plantar pressure data,
a device for computing a start timepoint and an end timepoint and intensity of at least one sequence of electrical pulses to apply to at least one of a nerve and a muscle of said patient using said at least one pair of electrodes based on said generated patient motion data and patient foot plantar pressure data.

According to another aspect, the invention relates to a method for computing a start timepoint and an end timepoint and an intensity of at least one sequence of electrical pulses to apply to at least one of a nerve and a muscle of a patient using at least one pair of electrodes, based on a gait phase information representative of a state of advancement of a patient in the gait cycle obtained using a personalized model of a gait cycle obtained from a device for training such as describe above, wherein said method comprises:
receiving, for said patient, patient motion data and patient foot plantar pressure data acquired during a plurality of gait cycles, said patient foot plantar pressure data being representative of a strength of a contact of at least one region of a foot with the ground, said patient foot plantar pressure data being acquired with at least one pressure sensor placed under said foot,
feeding said patient motion data and patient foot plantar pressure data to said personalized model of a gait cycle and output a gait phase information representative of a state of advancement of a patient in the gait cycle, and
computing said start timepoint and said end timepoint and said intensity of said at least one electrical pulse to apply to at least one of said nerve or said muscle of said patient using said at least one pair of electrodes.

In addition, the disclosure relates to a computer program product comprising instructions which, when is executed by a computer, cause the computer to carry out the method for computing a start timepoint and an end timepoint and an intensity of at least one electrical pulse to apply to at least one of a nerve and a muscle of a patient described above.

The present disclosure further pertains to a computer-readable storage medium comprising instructions which, when executed by a computer, cause the computer to carry out the method for computing a start timepoint and an end timepoint and an intensity of at least one sequence of electrical pulses to apply to at least one of a nerve and a muscle of a patient described above.

The present disclosure further pertains to a non-transitory program storage device, readable by a computer, tangibly embodying a program of instructions executable by the computer to perform a method for computing a start timepoint and an end timepoint and an intensity of at least one sequence of electrical pulses to apply to at least one of a nerve and a muscle of a patient, compliant with the present disclosure.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM).

### DEFINITIONS

In the present invention, the following terms have the following meanings:

The terms “adapted” and “configured” are used in the present disclosure as broadly encompassing initial configuration, later adaptation or complementation of the present device, or any combination thereof alike, whether effected through material or software means (including firmware).

The term “processor” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

“Machine learning (ML)” designates in a traditional way computer algorithms improving automatically through experience, on the ground of training data enabling to adjust parameters of computer models through gap reductions between expected outputs extracted from the training data and evaluated outputs computed by the computer models.

A “hyper-parameter” presently means a parameter used to carry out an upstream control of a model construction, such as a remembering-forgetting balance in sample selection or a width of a time window, by contrast with a parameter of a model itself, which depends on specific situations. In ML applications, hyper-parameters are used to control the learning process.

“Datasets” are collections of data used to build an ML mathematical model, so as to make data-driven predictions or decisions. In “supervised learning” (i.e. inferring functions from known input-output examples in the form of labelled training data), three types of ML datasets (also designated as ML sets) are typically dedicated to three respective kinds of tasks: “training”, i.e. fitting the parameters, “validation”, i.e. tuning ML hyperparameters (which are parameters used to control the learning process), and “testing”, i.e. checking independently of a training dataset exploited for building a mathematical model that the latter model provides satisfying results.

A “neural network (NN)” designates a category of ML comprising nodes (called “neurons”), and connections between neurons modeled by “weights”. For each neuron, an output is given in function of an input or a set of inputs by an “activation function”. Neurons are generally organized into multiple “layers”, so that neurons of one layer connect only to neurons of the immediately preceding and immediately following layers.

The above ML definitions are compliant with their usual meaning, and can be completed with numerous associated features and properties, and definitions of related numerical objects, well known to a person skilled in the ML field. Additional terms will be defined, specified or commented wherever useful throughout the following description.

A “pathological gait” refers to an abnormal walking pattern that is typically caused by an underlying medical condition or musculoskeletal disorder. This deviation from a normal walking pattern can be observed in the way a person moves their limbs, pelvis, and trunk during walking. A pathological gait may be characterized during the stance phase by abnormalities in weight distribution, foot positioning, timing of events, leading to an altered and potentially inefficient walking pattern. During the swing phase, a pathological gait may result in reduced clearance of the foot, uneven leg movement, or compensatory motions to navigate difficulties.

A “patient” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a disease. According to the invention, said patient has preferably a pathological gait (e.g. an abnormal manner of walking that deviates from the typical and healthy gait patterns).

A “subject” refers to a mammal, preferably a human. In one embodiment, a subject may be a “patient”, i.e. a warm-blooded animal, more preferably a human, who/which is used to collect data that may be used to construct a dataset for training a ML mathematical model. In the context of the invention, said subjects may be healthy patients with typical and healthy gait patterns or have a pathological gait.

A “motor point” of the muscle is the area in which the motor nerve endings enter (innervate) the muscle. Stimulation of motor point requires delivery of minimal electrical charge to excite the nerve endings by generating the action potentials in them that propagate to the muscle fibers and produce contraction of the muscle fibers.

### BRIEF DESCRIPTION OF THE DRAWINGS

FIG. 1 is a block diagram representing schematically a particular mode of a device for training a personalized model of a gait cycle compliant with the present disclosure,

FIG. 2 is a flow chart showing successive steps executed with the device for training a personalized model of a gait cycle of FIG. 1;

FIG. 3 is a block diagram representing schematically a particular mode of a device for computing a start timepoint and an end timepoint and an intensity of at least one electrical pulse to apply to at least one of a nerve and a muscle of a patient using the personalized model of a gait cycle obtained from the device represented in FIG. 1, compliant with the present disclosure;

FIG. 4 is a flow chart showing successive steps executed with the device computing a start timepoint and an end timepoint and an intensity of at least one electrical pulse to apply to at least one of a nerve and a muscle of a patient of FIG. 3;

FIG. 5 is a perspective view of the system for stimulating a muscle of a patient according to an embodiment,

FIG. 6 is a graphical representation of a comparison between predicted signals and actual signal obtained when the model is the generic model of a gait cycle or a personalized model of a gait cycle, and

FIG. 7 is a graphical representation of four graphs depicting gait percentages and gyroscope signals from the left and right legs, wherein the first graph and third graph represent a comparison of the gait percentages obtained with the first version of the model and the second version of the model and the ground truth (e.g. reference data obtained through manual or offline annotation processes) and wherein the second graph and fourth graph display the gyroscope signals (input motion data) obtained from the left foot and right food, respectively.

### DETAILED DESCRIPTION

The present description illustrates the principles of the present disclosure. It will thus be appreciated that those skilled in the art will be able to devise various arrangements that, although not explicitly described or shown herein, embody the principles of the disclosure and are included within its scope.

All examples and conditional language recited herein are intended for educational purposes to aid the reader in understanding the principles of the disclosure and the concepts contributed by the inventor to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein may represent conceptual views of illustrative circuitry embodying the principles of the disclosure. Similarly, it will be appreciated that any flow charts, flow diagrams, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, a single shared processor, or a plurality of patient processors, some of which may be shared.

It should be understood that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces.

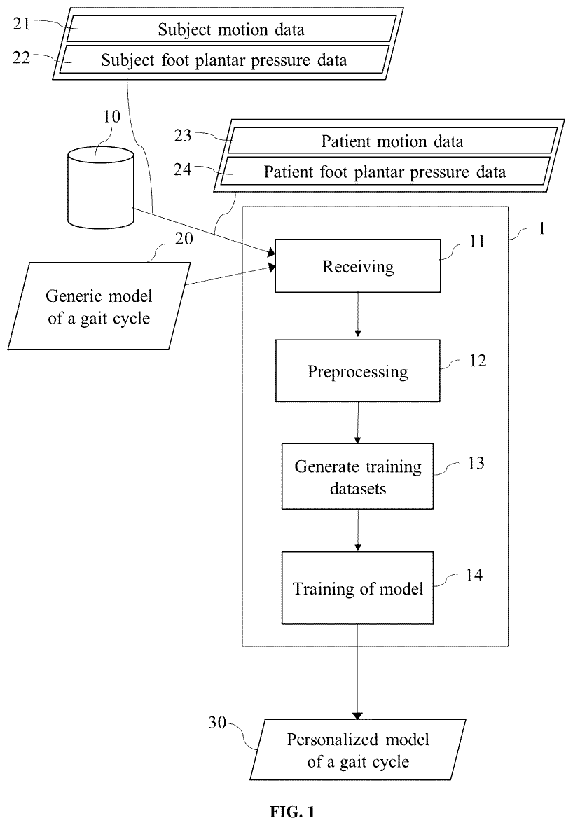

The present disclosure will be described in reference to a particular functional embodiment of a device 1 for training a personalized model of a gait cycle 30 configured to generate a gait phase information representative of a state of advancement of a patient in the gait cycle, as illustrated on FIG. 1.

The device 1 is adapted to output the personalized model of a gait cycle 30. This personalized model of a gait cycle 30 is configured to receive as input patient motion data 23 and patient foot plantar pressure data 24 acquired during a plurality of gait cycles and to output a gait phase information representative of a state of advancement of a patient in the gait cycle. In other words, based on these patient motion data 23 and patient foot plantar pressure data 24, the personalized model of a gait cycle 30 predicts the different phases of the patient's gait, even though said gait may be altered compared to a “normal gait”.

The training proposed in the present invention involves a first training phase implemented on a generic model of a gait cycle 20 based on a generic training dataset comprising motion 21 and foot plantar pressure 22 data relative to a plurality of gait cycles acquired from a plurality of subjects, and a second training phase that involves re-training the pre-trained generic model of a gait cycle obtained after the first training phase with a dataset comprising data relative to the patient generally suffering from a pathological gait so as to obtain the personalized model of a gait cycle 30.

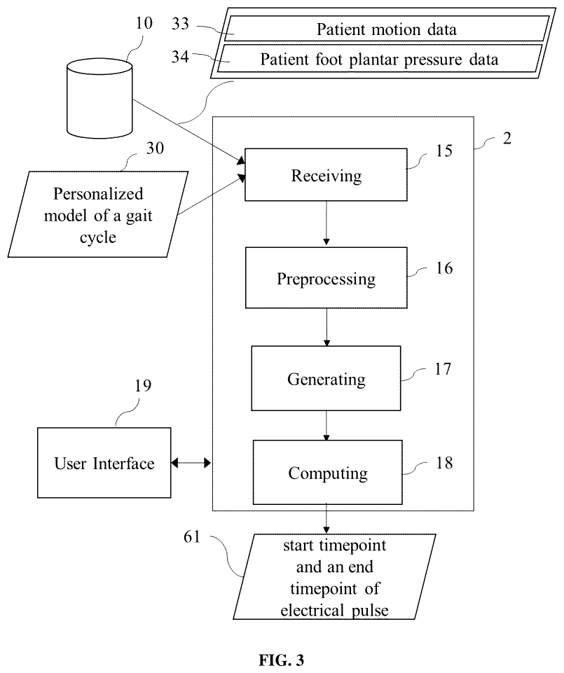

The device 1 for training the generic model of a gait cycle 20 is associated with a device 2, represented on FIG. 3, for computing a start timepoint and an end timepoint and an intensity of at least one sequence of electrical pulses to apply to at least one of a nerve and a muscle of a patient using at least one pair of electrodes 82, based on the gait phase information generated using the personalized model of a gait cycle 30 obtained from the device 1.

Though the presently described devices 1 and 2 are versatile and provided with several functions that can be carried out alternatively or in any cumulative way, other implementations within the scope of the present disclosure include devices having only parts of the present functionalities.

Each of the devices 1 and 2 is an apparatus, or a physical part of an apparatus, designed, configured and/or adapted for performing the mentioned functions and produce the mentioned effects or results. In alternative implementations, any of the device 1 and the device 2 is embodied as a set of apparatus or physical parts of apparatus, whether grouped in a same machine or in different, possibly remote, machines. The device 1 and/or the device 2 may have functions distributed over a cloud infrastructure and be available to users as a cloud-based service, or have remote functions accessible through an API.

The device 1 for training the personalized model of a gait cycle 20 and the device 2 for computing a start timepoint and an end timepoint and an intensity of at least one sequence of electrical pulses to apply to at least one of a nerve and a muscle of a patient may be integrated in a same apparatus or set of apparatus, and intended to same users.

For instance, device 1 and device 2 may be included in a rehabilitation apparatus configured to precisely predict the gait phase in which the patient is based only on motion and foot pressure data collected on said patient and provide an accurate muscle stimulation precisely timed to support and improve the patient's gait throughout the whole rehabilitation process. Thanks to device 1 and device 2, the rehabilitation apparatus may be capable of constantly adapting to subtle changes in gait dynamics and aid in gradually correcting the gait abnormalities as the patient progresses in his rehabilitation.

In other implementations, the structure of the device 2 may be completely independent of the structure of the device 1, and may be provided for other users. For example, the device 2 may include the personalized model of a gait cycle 30, wholly set from a previous personalization phase performed beforehand with the device 1.

In what follows, the modules are to be understood as functional entities rather than material, physically distinct, components. They can consequently be embodied either as grouped together in a same tangible and concrete component, or distributed into several such components. Also, each of those modules is possibly itself shared between at least two physical components. In addition, the modules are implemented in hardware, software, firmware, or any mixed form thereof as well. They are preferably embodied within at least one processor of the device 1 or of the device 2.

The device 1 comprises a module 11 for receiving, for each subject of a plurality of subjects, subject motion data 21 and subject foot plantar pressure data 22 previously acquired during a plurality of gait cycles and stored in one or more local or remote database(s) 10. The latter can take the form of storage resources available from any kind of appropriate storage means, which can be notably a RAM or an EEPROM (Electrically-Erasable Programmable Read-Only Memory) such as a Flash memory, possibly within an SSD (Solid-State Disk).

Alternatively, module 11 may be configured to receive as an input directly (i.e.; from a database 10) a generic training dataset previously constructed using the subject motion data 21 and subject foot plantar pressure data 22 and/or a training dataset previously constructed using the patient motion data 23 and patient foot plantar pressure data 24.

According to the present invention, subject motion data 21 and patient motion data 23 may be recordings obtained from at least one motion sensor such as IMUs (Inertial Measurement Unit), accelerometers, gyroscopes, magnetometers, Electromyography (EMG) sensors, joint angle sensors, cameras, infrared sensors, ultrasonic sensors and piezoelectric sensors used alone or in combination. Subject motion data 21 and patient motion data 23 may be obtained using the same sensors or different sensors.

The motion sensors may be placed on different localizations on the legs of the subjects/patient.

According to the invention, subject foot plantar pressure data 23 and patient foot plantar pressure data 24 may be obtained using pressure sensors positioned under the feet. The pressure sensors may be embedded directly in the shoes or in an insole that may be positioned inside the shoes. The pressure sensors may be configured to measure a pressure distribution applied by the foot on the ground during at least one gait cycle.

Recordings of subject motion data 21 and foot plantar pressure data 22 may be performed simultaneously on a treadmill with different speed conditions from 0.2 m/s to 1.2 m/s and on the ground. Advantageously, the subject motion data 21 and foot plantar pressure data 22 recorded on a same subject of the plurality of subjects are paired, meaning that there is a correspondence between the subject motion data 21 and foot plantar pressure data 22 as they have been recorded during the same plurality of gait cycles. This pairing allows the algorithm to learn the relationships, patterns, or correlations between the measurements obtained from the two types of sensors (e.g. pressure sensor and movement sensor). For instance, during the training of the algorithm, it learns how changes in measurements from one sensor correspond to changes in measurements from the other sensor, enabling it to make predictions or draw inferences when presented with new, unseen paired data from those sensors.

Additionally, module 11 may be further configured to receive subject environmental data representative of at least one parameter of an environment of said subject and patient environmental data representative of at least one parameter of an environment of said patient.

Subject environmental data and patient environmental data may be acquired using ultrasonic sensors and/or camera worn on said subject/patient.

Additionally, module 11 may be further configured to receive subject EMG data and patient EMG data, said subject EMG data and patient EMG data being representative of at least one of a muscle activity, a muscle spasticity or a muscle spasm.

EMG data may be acquired using EMG sensors worn on said subject/patient.

Additionally, EMG from the muscle may be recorded with the same dry electrodes used to stimulate the said muscle.

The device 1 further comprises optionally a module 12 for preprocessing the subject motion data 21 and/or subject foot plantar pressure data 22 and/or patient motion data 23 and/or patient foot plantar pressure data 24. Preprocessing may include data cleaning, normalization and temporal alignment, resampling, filtering and noise reduction.

Module 12 may also be used for postprocessing of the gait phase information representative of a state of advancement of a patient in the gait cycle outputted by the generic model of a gait cycle, the pre-trained generic model of a gait cycle, and/or the personalized model of a gait cycle. For instance, dilation and erosion (for ex. Of radius 3) operations may be performed to remove isolated parts of the gait phase information. For instance, isolated swings, where a swing phase occurs with a long previous contact, may be removed. This step helps eliminating false positives and ensures that gait phases are correctly linked to the appropriate contact points. Consecutive swings on the same side (e.g., left-left or right-right) may be evaluated, and only the swing with the higher movement quantity may be retained. This step accounts for situations where minor inconsistencies in the classification model's predictions may occur.

The device may comprise a module 13 for the construction of the generic training dataset and patient training dataset.

The generic training dataset generation may include a first step of detecting the toe-off events on the subject foot plantar pressure data 22 and/or on the subject motion data 21. Each gait cycle preferably includes a single toe-off event. As the subject foot plantar pressure data 22 and/or on the subject motion data 21 are recorded on a plurality of gait cycles, several toe-off events may be detected. Each toe-off event is then associated with a pre-determined state of advancement in the gait cycle. For example, each toe-off event may be associated with a percentage of 60% of advancement in the gait cycle.

The toe-off event is considered 60% of the cycle by convention. It does not mean that it corresponds to 60% of the gait time-period. The gait percentage is not linearly related to the time. It is used to properly set the stimulation sequence regardless of time that may vary between subjects. As a consequence, if the toe-off event occurs at a different time-point, for instance, at 70% (e.g. for example obtained from subjects suffering from a pathological gait), it's essential to still annotate it at the standard 60% for consistent learning. Annotating it at the standard position ensures the model learns from a consistent reference point, even if the actual event occurs at a different percentage within the gait cycle. This consistency aids in training the model effectively to predict and adapt stimulation patterns, despite variations caused by the pathology.

Moreover, other events may be detected such as the heel strike event and associated to the transition between two gait cycles. For example, each heel strike event may be associated with a percentage of 100% of advancement of a first gait cycle and of 0% of advancement with a second gait cycle that immediately follows the first gait cycle.

Similarly, the patient training dataset generation may also include a step of detection of the toe-off events on the patient foot plantar pressure data 23 and/or on the patient motion data 24. Each toe-off event may be associated with a pre-determined patient state of advancement in the gait cycle. For example, each toe-off event may be associated with a percentage of 60% of advancement in the gait cycle. If the toe-off event occurs at a different time-point, for instance, at 70% (e.g. because the patient suffers from a pathological gait), it's essential to still annotate it at the standard 60% for consistent learning. Annotating it at the standard position ensures the model learns from a consistent reference point, even if the actual event occurs at a different percentage within the gait cycle. This consistency aids in training the model effectively to predict and adapt stimulation patterns, despite variations caused by the pathology.

Moreover, other events may be detected such as the heel strike event and associated to the transition between two gait cycles. For example, each heel strike event may be associated with a percentage of 100% of advancement of a first gait cycle and of 0% of advancement with a second gait cycle that immediately follows the first gait cycle.

Annotation of the subject foot plantar pressure data 22 and/or on the subject motion data 21 may be performed manually or automatically by the at least one processor.

Additionally, module 13 may be further configured to construct a subject environmental dataset based on the subject environmental data and a patient environmental dataset based on the patient environmental data.

Additionally, module 13 may be further configured to construct a subject EMG dataset based on the subject EMG data and a patient EMG dataset based on the patient EMG data.

The device 1 may further comprise a module 14 configured to train the generic model of a gait cycle 20 using the generic training dataset constructed by module 13 (or received by module 11) to obtain a pre-trained generic model of a gait cycle.

Module 14 may further be configured to retrain the pre-trained generic model of a gait cycle using the patient training dataset so as to obtain the personalized model of a gait cycle 30.

Module 14 may further be configured to train the generic model of a gait cycle 20 using the subject environmental dataset and/or the subject EMG dataset.

The generic model of a gait cycle 20 may include a first block configured to perform a feature extraction and selection. The extracted features are then sent to a prediction block. The prediction block is configured to predict the shape of future gait cycles based on the data already acquired. The predicted data is then fed to a stimulation block, which relies on a functional electrical stimulation (FES) control algorithm. This FES control algorithm is configured to determine the pulse width, frequency, start timepoint and end timepoint of a sequence of pulses.

In a particular embodiment, the generic model of a gait cycle 20 architecture may be an Oscillatory Neural Network (ONN) configured to learn the features of the periodic signals received as input (e.g. motion data and foot plantar pressure data).

The generic model of a gait cycle 20 may be a deep Residual Network (ResNet) architecture comprising three blocks, each with three convolutional layers, Batch Normalization, and shortcut connections. The model uses global average pooling and ends with a Dense output layer using the ‘tanh’activation function for regression.

ResNet adds linear shortcut connections to facilitate training deeper networks while enabling effective learning of temporal features from the input data.

The patient motion data 23 and patient foot plantar pressure data 24 are used to re-train only the last fully connected layer of the pre-trained model of a gait cycle.

The pre-trained model of a gait cycle is then updated with the new calculated weights in order to obtain the personalized model of a gait cycle 30.

The pre-trained model of a gait cycle may also be re-trained using the patient environmental dataset and/or the patient EMG dataset.

Once the training is completed, module 14 is configured to output the obtained personalized model of a gait cycle 30. The personalized model of a gait cycle 30 may then by stored in one or more local or remote database(s) 10. The latter can take the form of storage resources available from any kind of appropriate storage means, which can be notably a RAM or an EEPROM (Electrically-Erasable Programmable Read-Only Memory) such as a Flash memory, possibly within an SSD (Solid-State Disk).

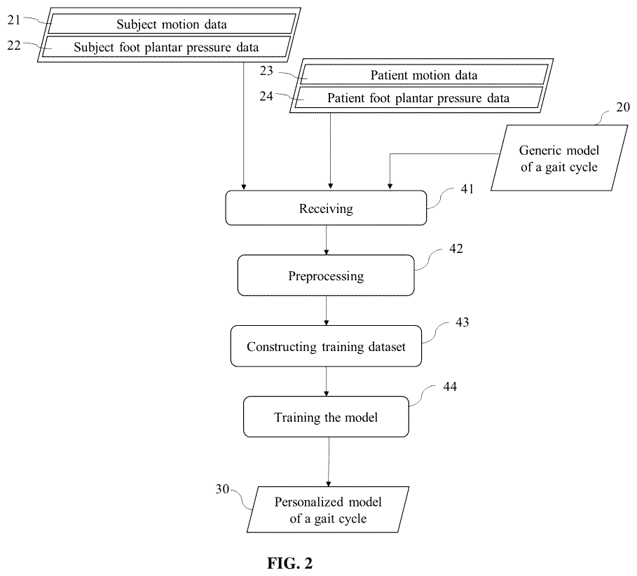

In its automatic actions, the device 1 may for example execute the following process (FIG. 2):
receiving for each subject of the plurality of subjects, subject motion data 21 and subject foot plantar pressure data 22 acquired during a plurality of gait cycles, and for said patient, patient motion data 23 and patient foot plantar pressure data 24 acquired during a plurality of gait cycles, said subject foot plantar pressure data 22 and patient foot plantar pressure data 24 being representative of a strength of a contact of at least one region of a foot with the ground, said patient foot plantar pressure data 22 and patient foot plantar pressure data 24 being acquired with at least one pressure sensor 22 placed under said foot (step 41),
optionally preprocessing the subject motion data 21 and/or subject foot plantar pressure data 22 and/or patient motion data 23 and/or patient foot plantar pressure data 24 (step 42),
constructing the generic training dataset using the subject motion data 21 and subject foot plantar pressure data 22 and constructing the patient training dataset based on the patient motion data 23 and patient foot plantar pressure data 24 (step 43),
training the generic model of a gait cycle 20 using said generic training dataset so as to obtain a pre-trained generic model of a gait cycle and retraining said pre-trained generic model of a gait cycle using said patient training dataset so as to obtain said personalized model of a gait cycle 30 (step 44).

The present invention also relates to a device 2 for computing a start timepoint and an end timepoint and an intensity of at least one sequence of electrical pulses to apply to at least one of a nerve and a muscle of a patient using at least one pair of electrodes, based on a gait phase information representative of a state of advancement of a patient in the gait cycle obtained using a personalized model of a gait cycle (30) obtained from the device 1, described above. The device 2 will be described in reference to a particular function embodiment as illustrated in FIG. 3.

The device 2 receives as input patient motion data 33 and patient foot plantar pressure data 34 acquired during a plurality of gait cycles. The patient motion data 33 and patient foot plantar pressure data 34 may be acquired using at least one motion sensor such as IMUs (Inertial Measurement Unit), accelerometers, gyroscopes, magnetometers, Electromyography (EMG) sensors, joint angle sensors, cameras, infrared sensors, ultrasonic sensors and piezoelectric sensors used alone or in combination. The motion sensors may be placed on different localizations on the legs of the subjects/patient.

According to the invention, patient foot plantar pressure data 33 may be obtained using pressure sensors positioned under the feet. The pressure sensors may be embedded directly in the shoes or in an insole that may be positioned inside the shoes. The pressure sensors may be configured to measure a pressure distribution applied by the foot on the ground during at least one gait cycle.

Recordings of patient motion data 33 and patient foot plantar pressure data 34 may be performed simultaneously during the daily movements of the patient or during a specific rehabilitation period during which the patient is asked to perform at least one exercise including activities such as walking, running, jumping or climbing stairs.

According to one embodiment, the patient motion data 33 and patient foot plantar pressure data 34 may be acquired using the system 1000 described below.

The device 2 is adapted to provide as output a start timepoint and an end timepoint and an intensity of at least one sequence of electrical pulses to apply to at least one of a nerve and a muscle of a patient using at least one pair of electrodes 82.

The device 2 comprises a module 15 for receiving the personalized model of a gait cycle 30 and the patient motion data 33 and patient foot plantar pressure data 34, stored in one or more local or remote database(s) 10. The latter can take the form of storage resources available from any kind of appropriate storage means, which can be notably a RAM or an EEPROM (Electrically-Erasable Programmable Read-Only Memory) such as a Flash memory, possibly within an SSD (Solid-State Disk). In some embodiments, the personalized model of a gait cycle 30 and all its parameters may have been previously generated by a system including the device 2 for training. Alternatively, the generic model of a gait cycle 20 and its training parameters may be received by the device 2 from a communication network.

Additionally, module 15 may be further configured to receive patient environmental data representative of at least one parameter of an environment of said patient. Patient environmental data may be acquired using ultrasonic sensors and/or camera worn on said patient.

Additionally, module 15 may be further configured to receive patient EMG data, said patient EMG data being representative of at least one of a muscle activity, a muscle spasticity or a muscle spasm. Patient EMG data may be acquired using EMG sensors worn on said patient.

The device 2 further comprises optionally a module 16 for preprocessing the patient motion data 33 and patient foot plantar pressure data 34. Preprocessing may include data cleaning, normalization and temporal alignment, resampling, filtering and noise reduction.

Module 16 may also be used for postprocessing of the gait phase information representative of a state of advancement of a patient in the gait cycle outputted by the personalized model of a gait cycle 30. For instance, dilation and erosion (for ex. Of radius 3) operations may be performed to remove isolated parts of the gait phase information. For instance, isolated swings, where a swing phase occurs with a long previous contact, may be removed. This step helps eliminating false positives and ensures that gait phases are correctly linked to the appropriate contact points. Consecutive swings on the same side (e.g., left-left or right-right) may be evaluated, and only the swing with the higher movement quantity may be retained. This step accounts for situations where minor inconsistencies in the classification model's predictions may occur. The aim is to clean the automatic annotation and to improve the quality of the annotated data for the training.

The device 2 may further comprises a module 17 configured to provide the patient motion data 33 and patient foot plantar pressure data 34 as input to said personalized model of a gait cycle 30 so as to generate a gait phase information representative of a state of advancement of a patient in the gait cycle.

Additionally, the patient environmental data and/or the patient EMG data may be provided as input to said personalized model of a gait cycle 30.

The device 2 may further comprises a module 18 configured to compute a start timepoint and an end timepoint and an intensity of at least one sequence of electrical pulses to apply to at least one of a nerve and a muscle of a patient using at least one pair of electrodes.

During its regular use, the device 2 outputs the start timepoint and an end timepoint and an intensity of a sequence of pulses that are used to stimulate the patient's nerves or muscles in real time to correct his pathological gait.

The device 2 may be used to stimulate both legs of said patient at a motor level (e.g. by stimulating motor nerves) to correct a pathological gait. Alternatively, device 2 may be used at a motor level on a paretic/paralyzed side of a patient and at a sensory level (e.g. by stimulating sensory nerves) on a healthy side of said patient to increase the inflow of sensory feedback to the brain and enhance brain plasticity. Alternatively, device 2 may be used at a sensory level on both legs to provide sensory biofeedback or feedback to the brain and increase brain excitability. Alternatively, device 2 may be used for stimulating the same leg both at a sensory level and at a motor level. To that end, at least one pair of electrodes dedicated to stimulation at a sensory level may be positioned on the leg and at least one second pair of electrodes dedicated to stimulation at a motor level may be positioned on the same leg. This helps limiting the potential onset of spasms (spasticity) while preserving the sensory inflow to the brain.

Advantageously, the EMG data allow to determine when a patient should be stimulated at a motor level, at a sensory level or at both motor and sensory levels.

The device 2 may interact with a user interface 19, via which information can be entered and retrieved by a user. The user interface 19 includes any means appropriate for entering or retrieving data, information or instructions, notably visual, tactile and/or audio capacities that can encompass any or several of the following means as well known by a person skilled in the art: a screen, a keyboard, a trackball, a touchpad, a touchscreen, a loudspeaker, a voice recognition system.

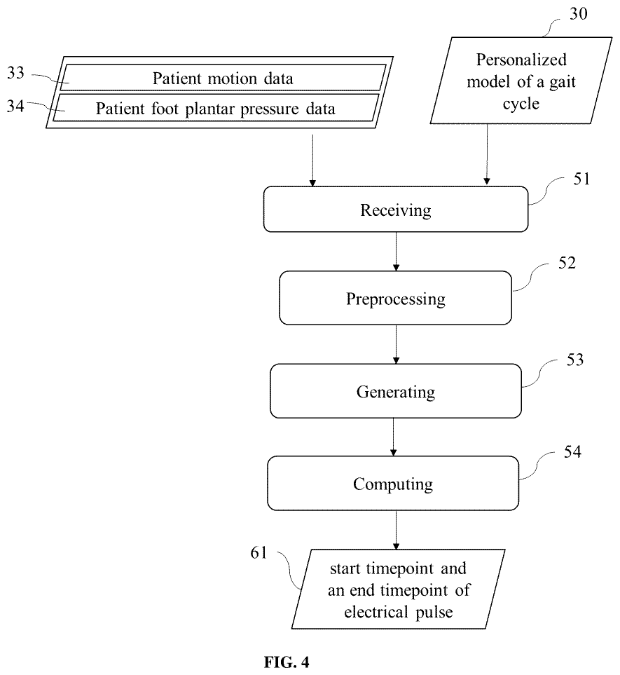

In its automatic actions, the device 2 may for example execute the following process (FIG. 4):
receiving the patient motion data 33 and patient foot plantar pressure data 34 (step 51),
preprocessing the patient motion data 33 and patient foot plantar pressure data 34 (step 52),
providing the preprocessed patient motion data 33 and patient foot plantar pressure data 34 to said personalized model of a gait cycle 30 and output a gait phase information representative of a state of advancement of a patient in the gait cycle (step 53),
computing a start timepoint and an end timepoint and an intensity of at least one sequence of electrical pulses to apply to at least one of a nerve and a muscle of a patient using at least one pair of electrodes 82 (step 54).

A particular apparatus may embody the device 1 as well as the device 2 described above. It corresponds for example to a workstation, a laptop, a tablet, a smartphone, or a head-mounted display (HMD).

That apparatus is suited to generation of segmentation mask and to related Machine Learning training. It comprises the following elements, connected to each other by a bus of addresses and data that also transports a clock signal:
a microprocessor (or CPU);
a graphics card comprising several Graphical Processing Units (or GPUs) and a Graphical Random Access Memory (GRAM); the GPUs are quite suited to image processing, due to their highly parallel structure;
a non-volatile memory of ROM type;
a RAM;
one or several I/O (Input/Output) devices such as for example a keyboard, a mouse, a trackball, a webcam; other modes for introduction of commands such as for example vocal recognition are also possible;
a power source; and
a radiofrequency unit.

According to a variant, the power supply is external to the apparatus.

The apparatus also comprises a display device of display screen type directly connected to the graphics card to display synthesized images calculated and composed in the graphics card. According to a variant, a display device is external to the apparatus and is connected thereto by a cable or wirelessly for transmitting the display signals. The apparatus, for example through the graphics card, comprises an interface for transmission or connection adapted to transmit a display signal to an external display means such as for example an LCD or plasma screen or a video-projector. In this respect, the RF unit can be used for wireless transmissions.

It is noted that the word “register” used hereinafter in the description of memories can designate in each of the memories mentioned, a memory zone of low capacity (some binary data) as well as a memory zone of large capacity (enabling a whole program to be stored or all or part of the data representative of data calculated or to be displayed). Also, the registers represented for the RAM and the GRAM can be arranged and constituted in any manner, and each of them does not necessarily correspond to adjacent memory locations and can be distributed otherwise (which covers notably the situation in which one register includes several smaller registers).

When switched-on, the microprocessor loads and executes the instructions of the program contained in the RAM.

As will be understood by a skilled person, the presence of the graphics card is not mandatory, and can be replaced with entire CPU processing and/or simpler visualization implementations.

In variant modes, the apparatus may include only the functionalities of the device 1, and not the learning capacities of the device 2. In addition, the device 1 and/or the device 2 may be implemented differently than a standalone software, and an apparatus or set of apparatus comprising only parts of the apparatus may be exploited through an API call or via a cloud interface.

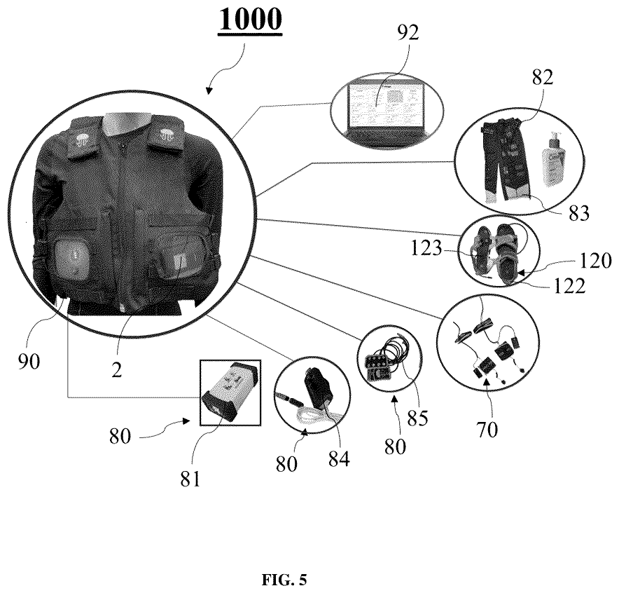

For instance, such apparatus may be the system 1000 for stimulating a muscle of a patient illustrated on FIG. 5. The system includes several elements.

A first element is a multi-channel electrostimulator 80 configured to send electric pulses to the muscles of the patient to stimulate them.

The electrostimulator 80 includes a portable electrical muscle stimulation (EMS) generator 81 that may be housed within a housing 90 such as a bag or a vest. The bag may be carried on the back or on the front or on one shoulder, or around the waist. Alternatively, the generator 81 may be housed in a pocket of an article of clothing 83, such as trousers.

The generator 81 may deliver a voltage comprised between 0 and 350 V and a current intensity comprised between 0 and 170 mA. The generator 81 is associated with an intensity variator 84 to deliver a pulse sequence with a pre-determined intensity, width and frequency.

The generator 81 may have several channels that can be each configured to deliver a different pulse sequence.

The generator 81 is in electrical connection 85 with a set of electrodes 82 configured to be positioned on the skin of the patient, either on the motor points of muscles, where nerve endings enter the muscle or on sensory receptors in the skin that are innervated or at sensory nerves endings.

The electrodes 82 may be connected to the generator 81 via conductive wires, electrical traces, conductive fibers, or a combination thereof. The conductive wires, electrical traces, conductive fibers, or a combination thereof can be embedded within the article of clothing 83, for instance in a layer of the article of clothing 83 or interwoven with fibers used to make the article of clothing 83.

The electrodes 82 may comprise several layers including a contact layer configured to attach to the patient's skin, a connector layer configured to be connected to the generator 81 and conductive layers positioned between the contact layer and connector layer. The contact layer can be made of a biocompatible polymeric layer. It may include an adhesive and/or a hydrogel. Alternatively, the contact layer may be dry and may require that a conductive gel or a hydrating lotion hydrates the skin surface of the patient.

The electrodes 82 may have different sizes and shapes such as round, oval, square or rectangle, or any other shape. The length and width of the electrodes may be comprised between 20 to 160 mm. Alternatively the diameter of the electrodes 82 is comprised between 20 to 80 mm.

In a preferred embodiment, the appropriate electrodes size is chosen so as not to exceed a power density of 0.1 Watts/cm². Therefore, the electrodes size is chosen depending on the maximum current intensity of the electrical pulse sent by the generator 81.

The electrodes 82 may be coupled to an inner surface of the article of clothing 83 by adhesives, clips, straps, hook-and-loop fasteners, stitches or a combination thereof. The electrodes 82 may be positioned such that when the patient puts the article of clothing 83 on, the contact layer of the electrodes 82 is automatically positioned in contact with the skin over the motor points, the sensory receptors or the sensory nerves endings that needs to be stimulated. Advantageously, the electrodes 82 can be detached from the article of clothing to allow replacing a defective electrode 82.

A second element of the system 1000 is at least one inertial measuring unit 70 and at least one pressure detection unit 120.

The inertial measuring unit 70 includes at least one motion sensor such as an accelerometer, a gyroscope, a magnetometer and/or a combination thereof. Advantageously, the inertial measuring unit 70 includes three motion sensors for each leg and one motion sensor for the hip. A first motion sensor may be positioned on the thigh, a second motion sensor may be positioned on the calf and a third motion sensor may be positioned on the foot. The motion sensors are configured to sense the three-dimensional movements of the leg throughout a gait cycle. The motion sensors may be embedded within the article of clothing 83, for instance in a layer of the article of clothing 83 or interwoven with fibers used to make the article of clothing 83.

The pressure detection unit 120 includes pressure sensors that may be positioned under the feet to sense the strength of a contact of the different parts of the patient's feet with the ground. The expression “strength of a contact” refers to the pressure exerted by a at least one region of one foot on the ground, the pressure being defined as a force per unit of area. Advantageously, the pressure sensors may be included in an insole or a sole of a shoe 122. For instance, the pressure detection unit 120 may include between 1 and 1000 sensors 123 distributed on the sole to collect data on the pressure applied, the position and the pressure changes when the patient is walking, running, jumping, climbing, descending, sitting or standing. In a preferred embodiment, there are five pressure sensors 123 per foot. Two pressure sensors may be positioned under the heel, on the medial and lateral side, and a third pressure sensor may be positioned under the big toe. The fourth and fifth sensors may be positioned under the metatarsal bones.

The pressure sensors 123 may be capacitive sensors, resistive sensors, piezoelectric sensors, piezoresistive sensors or a combination thereof. The pressure sensors 123 provide an electrical signal output, which is either a voltage or a current, that is proportional to the pressure exerted on said pressure sensors 123.

Advantageously, the system 1000 may include other sensors such as electromyography sensors to access muscle fatigue and movement intention or encoders positioned at the legs joints to access the angular position of the different parts on the legs.

For instance, the patient motion data 33 coming from the motion sensors and the patient foot plantar pressure data 34 coming from the pressure sensors may be used to determine the foot strike pattern, the foot inclination angle, the tibia angle, the hip flexion and extension, the trunk lean, the ankle inversion and eversion, the foot progression angle, the pelvic drop, the knee flexion and extension, the stride length, or the displacement of the center of mass, the speed of gait, the cadence, etc.

A third element of the system 1000 is device 2 for computing a start timepoint and an end timepoint of at least one sequence of electrical pulses to apply to at least one of a nerve and a muscle of a patient using the electrodes 82. The device 2 may be housed within the housing 90 with the generator 81. The device 2 may communicate with a control device 92 such as a tablet, a PC or a smartphone, hosting the user interface 19. The communication may be wireless, using Bluetooth, WiFi or other protocols. The control device 92 may be used to parameter the system 1000 and visualize information on the state of the system 1000 and on how the patient is faring.

The possible interactions with the control device 92 comprise the settings of the stimulation parameters for each muscle, intensities and timings; the commands for performing the personalization; the commands for starting and stopping the activity; and the visualization of instantaneous activation of muscles, of the percentage on the intensity variator, of signal graphs.

The device 2 is configured to receive the patient motion data 33 coming from the inertial measuring unit 70 and the patient foot plantar pressure data coming from the pressure detection unit 20. The generator 81 and the intensity variator 84 may be controlled by a user manipulating the device 2 to adjust the stimulation intensity and to send corresponding electrical pulses to the muscles using the electrodes 82. Connection between the device 2 and the generator 81 and intensity variator 84 and between the device 2 and the inertial measuring unit 70 and pressure detection unit 120 may be obtained using conductive wires, electrical traces, conductive fibers, or a combination thereof.

In use, when the patient walks, the inertial measuring unit 70 and pressure detection unit 120 continuously record information about said patient's gait. The data is then fed to an input layer of the personalized model of a gait cycle 30. This sequence is used to control the generator 81 and the intensity variator 84, by a manual manipulation by a user using the device 2, to deliver the pulse sequence to the muscles using the electrodes 82 to enhance gait rehabilitation of the patient.

Advantageously, the method may be used with an exoskeleton that may be actuated to guide the patient's movements, while the muscles are stimulated at the same time.

Ultrasonic sensors may be used to detect environmental data representative of at least one parameter of an environment of said patient such as a distance from an obstacle in the walking path and to adjust the stimulation pattern accordingly

Camera on the body may be used to detect obstacles in the walking path and to determine their size and shape in combination with distance detected by ultrasonic sensors, to adjust the stimulation pattern accordingly to avoid falls over the obstacle.

### EXAMPLES

The present invention is further illustrated by the following example.

### Example 1

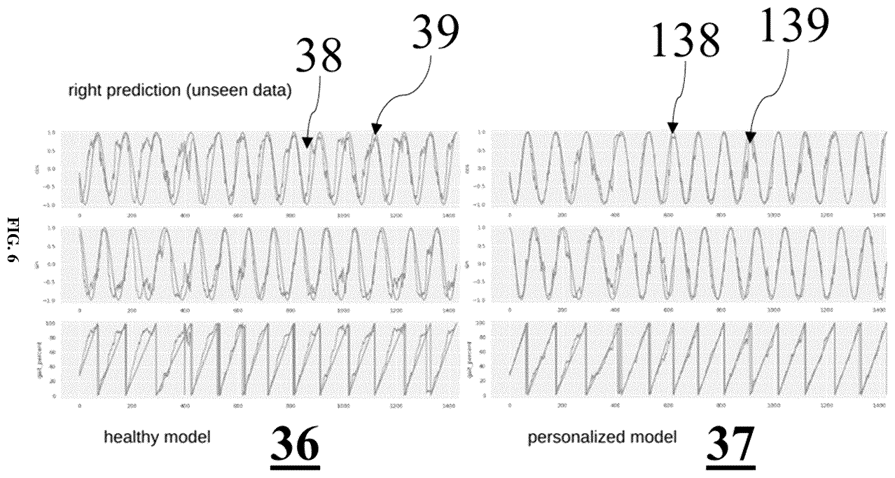

In this example, the generic model was trained with data coming from 20 healthy patients, with about 1 million window samples. As illustrated on FIG. 6, re-training the generic model of a gait cycle 20 to obtain a personalized model of a gait cycle 30 allows to greatly improve the prediction. Indeed, signals on the left 36 correspond to a comparison between predicted signals 38 and actual signal 39 obtained when the model is the generic model of a gait cycle 20 pre-trained with data coming from healthy patients.

Signals on the right 37 correspond to a comparison between predicted signals 138 and actual signal 139 obtained when the model is a personalized model of a gait cycle 30 re-trained with data coming from the patient suffering from a pathological gait.

We can observe that on the right 37, the predicted signals 138 and actual signal 139 are much better aligned than on the left 36. The prediction is therefore more accurate on the right 37.

### Example 2

In this example, generic model was trained with data coming from subjects with a pathological gait. More specifically, the subjects were a cohort of 44 hemiparetic subjects with about 1 million window samples.

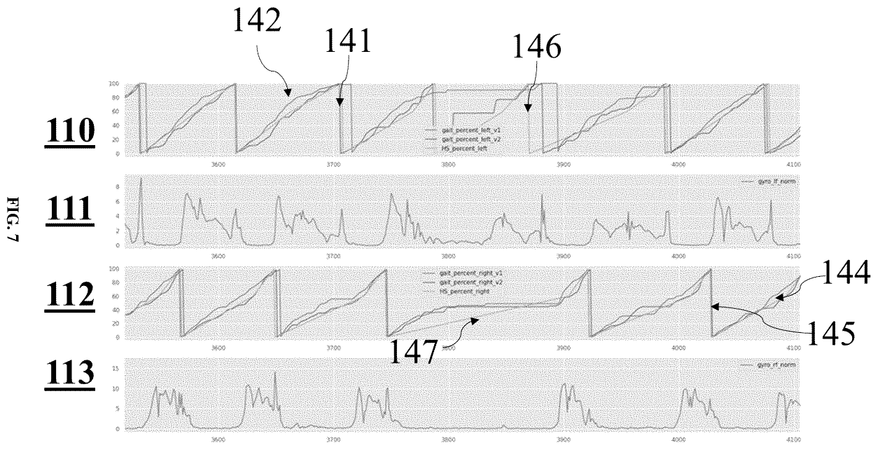

As illustrated on FIG. 7, the performance of the training on subjects with a pathological gait (called herein second version) was evaluated by comparing it with the results obtained using a model trained on subjects with a normal gait (called herein first version). This evaluation was conducted using five recordings, totaling 30 minutes of walking data. The mean squared error (MSE) was used as a quantitative measure to assess the improvement in performance.

FIG. 7 showcases four graphs depicting gait percentages and gyroscope signals from the left and right legs. The first graph 110 and third graph 112 represent a comparison of the gait percentages obtained with the first version of the model 142, 144, and the second version 141, 145, and the ground truth 146, 147 (e.g. reference data obtained through manual or offline annotation processes). The second graph 111 and fourth graph 113 display the gyroscope signals (input motion data) obtained from the left foot and right food, respectively.

In the first graph 110, a noticeable discrepancy is observed between the first version 142 and the ground truth 146, as the first version missed a step. Conversely, the second version 141 accurately captures the missed step, aligning more closely with the ground truth 146. Overall, the second model demonstrates improved accuracy, exhibiting a closer alignment to the ground truth than the first model.

The performance of the second version was further evaluated by comparing it with the first version on a patient who was not included in the training dataset. This evaluation was conducted using five recordings, totaling 30 minutes of walking data. The mean squared error (MSE) was used as a quantitative measure to assess the improvement in performance.

The comparison revealed that the second version of the model exhibited a significant improvement over the first version. On average, the MSE decreased by 38%. This reduction in MSE indicates that the second version achieved better accuracy in estimating gait phases compared to the first version. The improved performance can be attributed to the utilization of a larger and more diverse dataset, including those with different gait patterns and impairments.

The decrease in MSE means that a more precise alignment between the model's predictions and the ground truth gait phases is obtained for the patient's recordings. This enhancement in accuracy is crucial for the effective determination of gait phase transitions and the subsequent pattern of Functional Electrical Stimulation (FES) on muscles.

The observed improvement in performance highlights the importance of training the model on subjects'data and demonstrates the generalizability of the developed model across different subjects with gait impairments.

1-18. (canceled)

19. A device for training a personalized model of a gait cycle configured to generate a gait phase information representative of a state of advancement of a patient in the gait cycle, said device comprising:
at least one input configured to receive:
for each subject of a plurality of subjects, subject motion data and subject foot plantar pressure data acquired during a plurality of gait cycles,
for said patient, patient motion data and patient foot plantar pressure data acquired during a plurality of gait cycles,
said subject foot plantar pressure data and patient foot plantar pressure data being representative of a strength of a contact of at least one region of a foot with a ground, said subject foot plantar pressure data and patient foot plantar pressure data being acquired with at least one pressure sensor placed under said foot, at least one processor configured to:
generate a generic training dataset based on said subject motion data and subject foot plantar pressure data from the plurality of subjects,
generate a patient training dataset based on said patient motion data and patient foot plantar pressure data,
train a generic model of a gait cycle using said generic training dataset so as to obtain a pre-trained generic model of a gait cycle, and
retrain said pre-trained generic model of a gait cycle using said patient training dataset so as to obtain said personalized model of a gait cycle,
at least one output configured to provide said personalized model of a gait cycle.

20. The device according to claim 19, wherein generating said generic training dataset comprises, for each subject of the plurality of subjects, detecting on the subject foot plantar pressure data and subject motion data, a toe-off event for each gait cycle of the plurality of gait cycles, and associating said toe-off event with a pre-determined subject state of advancement in the gait cycle.

21. The device according to claim 20, wherein said pre-determined subject state of advancement in the gait cycle is a percentage of advancement in the gait cycle of 60%.

22. The device according to claim 19, wherein generating said patient training dataset comprises detecting on the patient foot plantar pressure data and patient motion data, a toe-off event for each gait cycle of the plurality of gait cycles, and associating said toe-off event with a pre-determined patient state of advancement in the gait cycle.

23. The device according to claim 22, wherein said pre-determined patient state of advancement in the gait cycle is a percentage of advancement in the gait cycle of 60%.

24. The device according to claim 19, wherein said generic model of a gait cycle is an oscillatory neural network.

25. The device according to claim 19, wherein subject motion data and subject foot plantar pressure data are acquired on subjects having a pathological gait.

26. A device for computing a start timepoint and an end timepoint and an intensity of at least one sequence of electrical pulses to apply to at least one of a nerve and a muscle of a patient using at least one pair of electrodes, based on a gait phase information representative of a state of advancement of a patient in the gait cycle obtained using a personalized model of a gait cycle obtained from a device according to claim 19, wherein said device comprises:
at least one input configured to receive, for said patient, patient motion data and patient foot plantar pressure data acquired during a plurality of gait cycles, said patient foot plantar pressure data being representative of a strength of a contact of at least one region of a foot with the ground, said patient foot plantar pressure data being acquired with at least one pressure sensor placed under said foot,
at least one processor configured to:
feed said patient motion data and patient foot plantar pressure data to said personalized model of a gait cycle and output a gait phase information representative of a state of advancement of said patient in the gait cycle, and
compute said start timepoint and said end timepoint and said intensity of said at least one sequence of electrical pulses to apply to at least one of said nerve and said muscle of said patient using said at least one pair of electrodes, and
at least one output configured to provide said start timepoint and end timepoint and intensity.

27. The device from claim 26, wherein said at least one input is further configured to receive patient environmental data representative of at least one parameter of an environment of said patient, said at least one processor being further configured to feed said patient environmental data to said personalized model of a gait cycle in order to adapt the computing of the start timepoint and end timepoint and of the intensity of the at least one sequence of electrical pulses as a function of the at least one parameter of the environment of said patient.

28. The device from claim 26, wherein said at least one parameter of an environment of said patient is at least one of a presence, a distance and a size of at least one obstacle present in said environment.

29. The device according to claim 26, wherein said at least one input is further configured to receive patient EMG data, said patient EMG data being representative of at least one of a muscle activity, a muscle spasticity and a muscle spasm, said at least one processor being further configured to feed said patient EMG data to said personalized model of a gait cycle in order to adapt the computing of the start timepoint and end timepoint and of the intensity of the at least one sequence of electrical pulses as a function of the patient EMG data.

30. The device according to claim 26, wherein said at least one input is further configured to receive patient EMG data, said patient EMG data being representative of at least one of a muscle activity, a muscle spasticity or a muscle spasm, said at least one processor being further configured to feed said patient EMG data to said personalized model of a gait cycle in order to determine if said at least one sequence of electrical pulses should be delivered to at least one of said nerve and said muscle at a motor level or at a sensory level.

31. The device from claim 30, wherein said at least one sequence of electrical pulses is delivered at a motor level when said patient EMG data exceeds a pre-determined value.

32. The device from claim 26, wherein feeding said patient motion data and patient foot plantar pressure data to said personalized model of a gait cycle comprises using a rolling window.

33. A system for stimulating a muscle of an individual, comprising:
a multi-channel electrostimulator comprising at least one pair of electrodes,
at least one inertial measuring unit configured to generate said patient motion data and at least one pressure detection unit configured to generate said patient foot plantar pressure data,
a device according to claim 26 for computing a start timepoint and an end timepoint and an intensity of at least one sequence of electrical pulses to apply to at least one of a nerve and a muscle of said patient using said at least one pair of electrodes based on said generated patient motion data and patient foot plantar pressure data.

34. (canceled)

35. (canceled)
